(19) United States
(12) Patent Application Publication
(10) Pub. No.: US 2026/0185032 A1
(43) Pub. Date: Jul. 2, 2026

(54) PROCESS FOR STIMULATING THE LIFE CYCLE OF YEAST BY MEANS OF AN ACOUSTIC BIOREACTOR AND RELATED MONITORING METHOD

(71) Applicant: YEASTIME START UP S.R.L., Guidonia Montecelio (RM) (IT)

(72) Inventors: Luca BUCCARELLO, Guidonia Montecelio (RM) (IT); Alessandro CONTALDO, Guidonia Montecelio (RM) (IT); Pierfrancesco MAZZOLINI, Guidonia Montecelio (RM) (IT); Federico ORTENZI, Guidonia Montecelio (RM) (IT)

(21) Appl. No.: 18/727,946

(22) PCT Filed: Dec. 13, 2022
(86) PCT No.: PCT/IB2022/062111

§ 371 Date: Jul. 10, 2024

(30) Foreign Application Priority Data
Jan. 17, 2022 (IT) 102022000000608

(51) Int. Cl. C12M 1/42; C12C 11/00; C12M 1/34; C12M 1/36; C12N 1/18; C12R 1/865

(52) U.S. Cl. CPC C12M 35/04; C12C 11/00; C12M 41/12; C12M 41/26; C12M 41/32; C12M 41/34; C12M 41/48; C12N 1/18; C12R 2001/865

(57) ABSTRACT

A process for stimulating the life cycle of yeast by means of an acoustic bioreactor, characterized in that it reduces fermentation time by increasing the yield of aroma-generating metabolites, through the activation of beta-D-glucosidase by means of low energy ultrasound, using an acoustic wave generator and a transducers' system; wherein fermentation inside said acoustic bioreactor is monitored by a plurality of sensors according to the needs of the client and to the fermentation taken into consideration.

### FIELD OF THE INVENTION

The invention relates to an innovative technique for stimulating yeast fermentation by means of a band of sound waves, through ultrasound range and specific irradiation times, optimizing the fermentation conditions obtaining an improvement in yield for large-scale productions.

### PRIOR ART

Our planet suffers from an intense rate of extraction and consumption of its natural resources due to human activities. There is therefore a need to look for effective alternatives to supply nutrients, chemicals and energy, from methodologies that have limited impact on the environment. In this perspective, the “Green Deal” program developed by the European Commission outlines the way forward in finding new strategies to support economic growth, decoupling it from the consumption of resources. Through the introduction of a new regulatory framework on the circular economy, biodiversity and innovation, it is necessary to focus on reducing the human impact on natural resources.

Fermentation is an anaerobic process, in which enzymes or microorganisms carry out the extraction of energy from carbohydrates, it is generally associated with the production of carbon dioxide. Although fermentation is involved in countless industrial processes, it has remained anchored to traditional methods and lacks specific control over times, energy consumption and related environmental impacts.

Yeast cultures are used for many different areas of industrial production, such as food and pharmaceuticals. This is a rapidly developing field of research and investment, although there are some problems to overcome concerning genetically modified organisms, crop management and their economic viability.

During yeast culture, parameters such as temperature and nutrients could be set and modified according to the yeast fermentation state, to optimize fermentation and reduce consumption. Today's technology, as demonstrated by the validation of the patent CN112240876, released on Jan. 19, 2021, reports a methodology for monitoring fermentation processes using infrared that has nothing to do with optimizing fermentation processes and saving energy that today's market needs.

The object of the patent is therefore to propose the use of a wave cycle administered via an acoustic bioreactor which, through the transduction of an electrical signal into a mechanical signal in the form of ultrasound, can stimulate the life cycle of the yeast and encourage fermentation, reducing times, consumption and energy waste common in the industrial sector; said acoustic bioreactor being further manageable remotely via wireless connections.

### DESCRIPTION OF THE INVENTION

According to the present invention, a certification system of the educational path of a student is implemented, which effectively solves the problems outlined above.

It has been demonstrated that, depending on the features of the sound wave and the properties of the target organism, there is a wide range of effects exerted by sound radiation. The sound vibration from mechanical stimulus is transmitted as a cellular and metabolic signal, so as to influence life at different levels.

The proposed signal transduction mechanism involves the activation of cellular mechanoreceptors, which cause membrane depolarization, action potential generation, and induction of secondary metabolism in the target organism.

Said study was applied to the fermentation of yeast, microalgae and other fermenting microorganisms, implementing a bioreactor with an integrated acoustic component, adapted to define the entire machine as an acoustic bioreactor.

By virtue of the cycle of ultrasound waves applied in the acoustic bioreactor, the fermentation time has been decreased, increasing the yield of aromatic metabolites, through the activation of the beta-d-glycosidase present in the microorganism, through low energy ultrasounds.

The present acoustic bioreactor exploits an ultrasonic wave generator and a transducer system to optimize the fermentation, which is further monitored through the use of a plurality of sensors which vary according to the customer's needs and the fermentation parameters under consideration.

A signal generator is adapted to generate the square rectangular or sinusoidal wave, operating in the low frequency ultrasound range (<100 kHz); the emitted signal must necessarily be amplified due to the resulting low power of the output signal (<10 W/L).

A power driver that acts as an amplifier is furthermore installed externally to the acoustic bioreactor, in order to process the output signal, amplifying it.

In order to extrapolate the spectrum of the signal sent by said signal generator, a hydrophone is installed inside said acoustic bioreactor.

A transducer is instead adapted to convert the electric signal into a vibration signal; said vibration signal includes a cycle of mechanical waves with the same frequency, of the rectangular or sinusoidal type, which may be irradiated inside the fermented substance, all integrated in the fermenter, externally or internally to the structure.

A microcontroller is installed to manage the power driver, so that the signal is transmitted with a duty cycle with Ton (irradiation time) that varies from 0.5 s to 8 s with respect to a Ttot (total period time) that varies from 10 s to 60 s, given that the irradiation period does not occur for the entire duration of fermentation but only in the logarithmic exponential increase step which goes from 10 h to 70 h in S. cerevisae and depends on the type of microorganism. In this step, the device goes to act and irradiate from 5% to 100% of the aforementioned time interval.

The frequency of the ultrasounds is adapted to vary in a range between 19 kHz and 99.99 kHz, while the irradiation power of said low power ultrasounds varies within a range between 200 mW/I and 1000 mW/I.

A plurality of probes and sensors are further installed inside the acoustic bioreactor object of the invention.

In fact, a probe is installed inside the fermentation chamber to monitor the temperature of the fermented product.

A plurality of sensors, of the chemo-conductive type, are instead adapted to monitor the gases emitted from the fermentation process.

In one embodiment thereof, the sensors dedicated to monitoring the gases, inside the fermentation chamber, are electrochemical sensors such as the Lambda probe.

In order to monitor the Plato, by virtue of which it is possible to obtain the density of the fermented product, a refractometer is used, in order to understand the progress of the fermentation.

A Clark electrode is instead included inside the fermentation chamber to monitor the pH of the fermented product.

The spectrum of absorption, refraction and reflection of the fermented substances also represents an important aspect to monitor in this field, for this reason a spectrophotometer external to the fermentation chamber is used.

A microcontroller is therefore adapted to interact with the plurality of probes and sensors mentioned above, in order to extrapolate the data, sending them to a server to which the user, owner of the acoustic bioreactor, can connect to check the state of the fermentation in real time.

The entire system obtains energy from a power supply circuit directly connected to the acoustic bioreactor in question.

By way of non-limiting example, said acoustic bioreactor is further used in the cultivation of cell cultures belonging to each taxon of living organisms for the production of biocompounds, secondary metabolites, and biomass/organoids.

The use of the stimulation process, in one embodiment thereof, also occurs in the creation of a unique beer aroma despite starting from the same yeast strain and the same malt.

A monitoring method of the acoustic bioreactor proves to be essential for retrieving and comparing the plurality of information collected by the sensors present inside said acoustic bioreactor.

These sensors are connected to a central management core to which each user can connect to read and check the information, using any previously registered device. All information obtained from these sensors passes from a cloud, which facilitates remote reading by each user.

Registration, on the dedicated web portal, is the first step that every user in possession of an acoustic bioreactor must take; the information concerning the readout of said sensors is available on a dedicated web portal due to the use of a REST API located a central server connected to the Wide Area Network, hosting the management core; wherein, said API ensures the possibility of loading the plurality of data collected from the sensors, on the dedicated portal.

Subsequently, the user mush log in to access the reserved area, by using the credentials previously inputted in the registration step.

The redemption of possession of the acoustic bioreactor takes place by entering, within the area dedicated to the user, a unique code present on the acoustic bioreactor; in one embodiment thereof, said redemption of possession takes place by scanning a QR code with a common smartphone.

The user then chooses the Wi-Fi to connect the acoustic bioreactor to, in order to facilitate fermentation monitoring.

The user will then be able to remotely modify the taste and aroma of the fermented product based on the type of production, using the previously described ultrasound technology. By way of non-limiting example, said modification of the taste and aroma of the fermented product takes place completely automatically, once the acoustic bioreactor has been synchronized within the user's reserved area, given that the user may have previously saved his/her preferences in his/her reserved area.

Finally, through the dedicated web portal, a common registered user can view, in real time, the sensor data sent by the acoustic bioreactor, as well as consult the temporal graphs of the data present in the database of the web portal.

The advantages offered by the present invention are clear in the light of the above description and will be even more apparent from the accompanying figures and the detailed description.

### DESCRIPTION OF THE FIGURES

The invention will hereinafter be described in at least a preferred embodiment thereof by way of non-limiting example with the aid of the accompanying figures, in which:

FIG. 1 shows the flow chart underlying the process of stimulating the life cycle of the yeast object of the invention.

FIG. 2 shows the flow chart underlying the method of the invention, connected to the monitoring of yeast fermentation inside the acoustic bioreactor.

### DETAILED DESCRIPTION OF THE INVENTION

The present invention will now be described purely by way of non-limiting or binding example with the aid of the figures, which illustrate some embodiments relative to the present inventive concept.

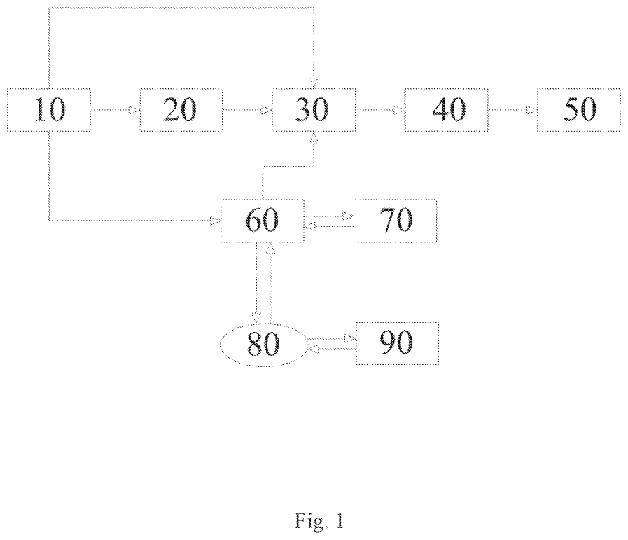

With reference to FIG. 1, the operating system of the stimulation process 50 of the yeast life cycle is illustrated and, by way of example, the process and setting applied to the acoustic bioreactor object of the invention are reported.

The Duty cycle used, in order to avoid a negative effect in the irradiation and to optimize the effectiveness of the treatment, provides for the irradiation of the crop intermittently, using a cycle of 6.25% (device 1 switch-on time second every 16 seconds).

The tests carried out assert that the radiated signal must be radiated from 0.5 s to 8 s (Ton) for a total time interval ranging from 10 s to 60 s (Ttot).

The selected frequency, on the other hand, is set according to the chemical-physical, biochemical, genetic and epigenetic features of the target microorganism, in fact the pitch of the wave is determined by the parameters mentioned above, including the cell diameter of Saccharomyces cereviasae, and consequently the wavelength chosen in the administration cycle is set.

The selected frequencies are:
25 KHz with an error of +/31 KHz
40 KHz with an error of +/−3 KHz

The preferable frequency and object of the invention is the ultrasound frequency, using a range that varies from 19 kHz to 99.99 kHz.

The low power used is indicated to avoid the cavitation phenomenon and prevent cell death.

The powers used are:
590 mW/l with an error of +/−10 mW/l
300 mW/l with an error of +/−10 mW/l

The irradiation power varies in a range from 200 mW/l to 1000 mW/l.

Irradiation in the logarithmic exponential growth phase which ranges from 10 h to 70 h in S. cerevisae and depends on the type of microorganism.

The LOG phase for each fermentation process generally begins after 10 h and ends after 72 h, in this phase the project goes into action and irradiates in this time interval from 5% to 100%.

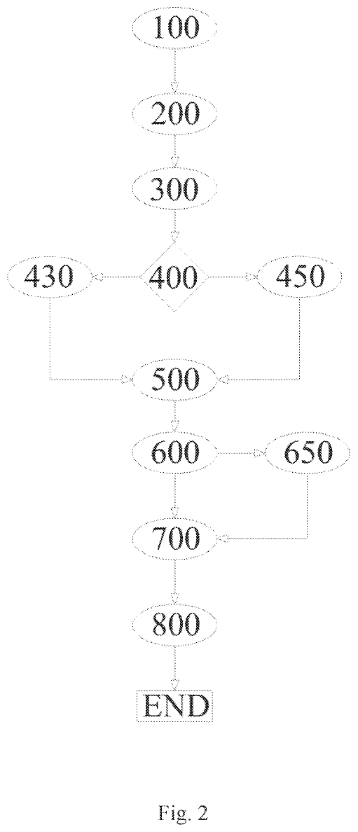

With reference to FIG. 2, the flow diagram suitable for explaining the steps described by the present monitoring method of the acoustic bioreactor for the stimulation 50 of the yeast life cycle is illustrated. In order to obtain the correct functioning of the monitoring method, the following steps have been identified:
registration 100 on the dedicated web portal, of each user 90 who owns an acoustic bioreactor, or of anyone who has been entrusted with the management of an acoustic bioreactor; wherein, information concerning the readout of said sensors 70 is available on a dedicated web portal due to the use of a REST API and a central server connected to the Wide Area Network, hosting the management core; wherein, said API ensures the possibility of loading the plurality of data collected from the sensors 70, on the dedicated portal;
login 200 of the user 90 to access the reserved area, by using the credentials previously inputted during registration;
selection of the Wi-fi 300 to which the acoustic bioreactor should be connected, in order to facilitate monitoring of the fermentation process by the user 90;
redemption of possession 400 of the acoustic bioreactor by inserting 430 in the dedicated area of the user 90 previously registered on the web portal, a unique code present on the acoustic bioreactor;
selection of the product to be processed 500;
modification 600, performed by the user 90, of the taste and aroma of the fermented product, depending on the kind of production, by using the above-described ultrasound technology;
real-time visualization 700 of sensor data sent by the acoustic bioreactor;
examination 800 of time diagrams of the data present in the database of the web portal.

Finally, it is clear that modifications, additions or variants may be made to the invention described thus far which are apparent to those skilled in the art, without departing from the scope of protection that is provided by the appended claims.

1. A process (50) for stimulating the life cycle of yeast by means of an acoustic bioreactor, characterized in that it reduces fermentation time by increasing the yield of aroma-generating metabolites, through the activation of beta-D-glucosidase by means of low energy ultrasound, using an acoustic wave generator and a transducers' system (40); wherein fermentation inside said acoustic bioreactor is monitored by a plurality of sensors (70) according to the needs of the user (90) and to the fermentation taken into consideration; said acoustic bioreactor comprising:
at least a power supply circuit (10) suited to supply energy to the present stimulation process (50) founded on an acoustic bioreactor;
at least a signal generator (20) for generating a rectangular or sinusoidal wave, operating in the low frequency ultrasound range, said signal being necessarily amplified due to the resulting low power of the output signal;
at least a power driver acting as an amplifier (30), for processing the output signal by amplifying it;
at least a transducer (40) for converting the electric signal into a vibration signal; said vibration signal including a sequence of mechanical waves with the same frequency and of the rectangular or sinusoidal type, which are irradiated inside the fermented substance;
at least a microcontroller (60) suited to interact with the plurality of probes and sensors (70), in order to extract the data and send them to a server to which a user (90), owner of the acoustic bioreactor, is connectable to verify the state of the fermentation process;
said microcontroller (60) also having the task of governing said power driver which acts as an amplifier (30), in such a way that the signal is transmitted with a duty cycle where Ton varies from 0.5 sec to 8 sec with respect to a Ttot comprised in the interval of 10 sec to 60 sec, since the irradiation period does not cover the whole duration of fermentation but only the logarithmic exponential increase step from 10 h to 70 h in S. cerevisiae and depends from the kind of microorganism; said irradiation occurring in a time interval from 5% to 100%;
at least a digital-type probe, installed in the fermentation chamber, for monitoring the temperature of the fermented substance;
a plurality of sensors, of the chemosensor type, installed inside the fermentation chamber, for monitoring the emitted gases of the fermentation process;
at least a refractometer for monitoring the Plato, by which the density of the fermented product is obtainable in order to recognize whether the fermentation process is proceeding well;
at least a Clark electrode for monitoring the pH of the fermented product;
at least an external spectrophotometer, for monitoring the optical properties of the fermented product;
at least a hydrophone, suited to extract the spectrum sent by said signal generator;
at least an online cloud (80) for collecting the data extracted by said sensors, in order to engage and keep updated the user (90) about said stimulation process (50); wherein said cloud (80) renders the obtained information remotely consultable using any kind of electronic device owned by the user (90).

2. A process (50) for stimulating the life cycle of yeast by means of an acoustic bioreactor, according to claim 1, characterized in that said digital-type probe for temperature monitoring of the fermented product, cooperates with a thermal imaging camera and with a plurality of thermistors of the NTC or PTC type, in order to optimize said monitoring.

3. A process (50) for stimulating the life cycle of yeast by means of an acoustic bioreactor, according to claim 1, characterized in that electrochemical sensors like the lambda probe are used to monitor the presence of gases inside the fermentation chamber.

4. A process (50) for stimulating the life cycle of yeast by means of an acoustic bioreactor, according to claim 1, characterized in that said duty cycle has a signal varying from 0.5 to 8 seconds in a time interval from 10 to 60 seconds.

5. A process (50) for stimulating the life cycle of yeast by means of an acoustic bioreactor, according to claim 1, characterized in that the frequency of ultrasound varies within a range from 19 kHz to 99.99 kHz.

6. A process (50) for stimulating the life cycle of yeast by means of an acoustic bioreactor, according to claim 1, characterized in that the irradiation power of said low power ultrasound varies within a range from 200 mW/l and 1000 mW/l.

7. Use of the process (50) for stimulating the life cycle of yeast by means of an acoustic bioreactor, according to claim 1, in the cultivation of cell cultures belonging to any taxon of living organisms, for the production of biocomposites, secondary metabolites, and biomasses/organoids.

8. Use of the process for stimulating the life cycle of yeast by means of an acoustic bioreactor, according to claim 1, in the production of a single beer aroma despite starting from the same yeast strain and the same malt.

9. A method of monitoring the acoustic bioreactor for the stimulation (50) of the life cycle of yeast, characterized in that the plurality of sensors (70) located inside the acoustic bioreactor are connected to a central management core to which each user (90) can connect himself/herself, in order to find information concerning the operation of the acoustic bioreactor, by using any kind of electronic device; wherein the final user (90) is thereby put in the condition to have real-time access to the monitoring process of the internal parameters, by using any kind of smartphone or computer; said method of monitoring including the following steps:
registration (100) on the dedicated web portal, of each user (90) who owns an acoustic bioreactor, or of anyone who has been entrusted with the management of an acoustic bioreactor; wherein, information concerning the readout of said sensors is available on a dedicated web portal due to the use of a REST API and a central server connected to the Wide Area Network, hosting the management core; wherein, said API ensures the possibility of loading the plurality of data collected from the sensors (70), on the dedicated portal;
login (200) of the user (90) to access the reserved area, by using the credentials previously inputted during registration;
selection of the Wi-fi (300) to which the acoustic bioreactor should be connected, in order to facilitate monitoring of the fermentation process by the user (90);
redemption of possession (400) of the acoustic bioreactor by inserting (430) in the dedicated area of the user (90) a unique code present on the acoustic bioreactor;
selection of the product to be processed (500);
modification (600), performed by the user (90), of the taste and aroma of the fermented product, depending on the kind of production, by using the above-described ultrasound technology;
real-time visualization (700) of sensor data sent by the acoustic bioreactor;
examination (800) of time diagrams of the data present in the database of the web portal.

10. A method of monitoring the acoustic bioreactor for the stimulation (50) of the life cycle of yeast according to claim 9, characterized in that said modification of the taste and aroma of the fermented product occurs in a fully automatic way (650), after the synchronization of the acoustic bioreactor with the reserved area of the user (90), because the user has previously saved his/her preferences in the respective reserved area.

11. A method of monitoring the acoustic bioreactor for the stimulation (50) of the life cycle of yeast according to claim 9, characterized in that said redemption of possession occurs by scanning (450) a QR code on the acoustic bioreactor by means of a common smartphone connected to the web portal.
